(19) United States
(12) Patent Application Publication
(10) Pub. No.: US 2019/0111191 A1
(43) Pub. Date: Apr. 18, 2019

(54) EVACUATED DRAINAGE CONTAINER

(71) Applicant: pfm Medical, Inc., Carlsbad, CA (US)

(72) Inventors: Jiaye Jho, Carlsbad, CA (US); Tamara Varcasia, Carlsbad, CA (US); Daniel Hicks, Carlsbad, CA (US); Alex Novichenok, Carlsbad, CA (US)

(21) Appl. No.: 15/787,410

(22) Filed: Oct. 18, 2017

(51) Int. Cl. A61M 1/00; B65D 88/42

(52) U.S. Cl. CPC A61M 1/0003; A61M 1/0023; B65D 2543/00009; A61M 1/0058; B65D 88/42

(57) ABSTRACT

A pre-evacuated container for medical purposes includes an outer body that forms an internal cavity, wherein in a default state the internal cavity is at a vacuum pressure relative to atmosphere, and a fluid interface on the outer body that connects to a drainage tube. A pressure indicator on the outer body is adapted to provide an indication as to a fluid pressure state of the internal cavity. An outlet assembly can be actuated to form an opening between atmosphere and the internal cavity, wherein the outlet assembly can be actuated without damaging the outer body.

### BACKGROUND

Pre-evacuated reservoirs for medical purposes, such as in the form of Redon flasks, are used for drainage and storage of body fluids such as in a post-operative wound drainage scenario. The reservoirs are often in the form of a bottle or flask formed of a rigid material such as plastic. Connection portions, such as one or more drainage conduits, are coupled to an interior cavity of the reservoir. In addition, the reservoir can also include a vacuum or pressure indicator to provide an indication as to the pressure state of the reservoir's internal cavity.

The interior cavity of the reservoir can be subjected, under sterile conditions, to a negative pressure of more than 90 kPa relative to atmosphere. When the interior cavity is attached to a wound drainage tube (which is coupled to a body wound cavity), a corresponding pressure difference arises between the wound cavity, in which one end of the drainage tube is located, and the environment. This pressure difference causes fluid to drain from the wound cavity into the reservoir via the wound drainage tube.

It can be difficult and cumbersome to open a reservoir for drainage and/or disposal of the reservoir contents after the reservoir has been filled with fluid. In a conventional reservoir, a user must cut open the reservoir to release its fluid. This process can be messy and can expose the user to bio-waste.

In view of the foregoing, there is a need for improved devices and methods for opening a pre-evacuated drainage reservoir, such as a Redon bottle.

### SUMMARY

Disclosed herein is a body cavity or wound drainage system having a reservoir into which body fluids can drain. The reservoir includes a convenient outlet assembly that can be opened or otherwise actuated to remove fluids or other contents from the reservoir such as when the reservoir is filled to capacity. The outlet assembly is configured such that the reservoir can be opened without cutting or otherwise damaging the reservoir. The material of the reservoir does not have to be cut, torn, bent or otherwise plastically deformed in order to open the reservoir.

In one aspect, there is disclosed a pre-evacuated container for medical purposes, comprising: an outer body that forms an internal cavity, wherein in a default state the internal cavity is at a vacuum pressure relative to atmosphere; a fluid interface on the outer body that connects to a drainage tube such that fluids can be evacuated into the container from a body cavity via the drainage tube; a pressure indicator on the outer body, the pressure indicator adapted to provide an indication as to a fluid pressure state of the internal cavity; and an outlet assembly on the outer body, wherein the outlet assembly can be actuated to form an opening between atmosphere and the internal cavity, wherein the outlet assembly can be actuated without damaging the outer body.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

The details of one or more variations of the subject matter described herein are set forth in the accompanying drawings and the description below. Other features and advantages of the subject matter described herein will be apparent from the description and drawings, and from the claims.

FIG. 1 shows a wound drainage system.

FIG. 2 shows a container of a wound drainage system wherein the container has an outlet assembly for emptying contents of the container.

FIGS. 3-8 show additional embodiments of the container.

### DETAILED DESCRIPTION

Before the present subject matter is further described, it is to be understood that this subject matter described herein is not limited to particular embodiments described, as such may of course vary. It is also to be understood that the terminology used herein is for the purpose of describing particular embodiments only, and is not intended to be limiting. Unless defined otherwise, all technical terms used herein have the same meaning as commonly understood by one skilled in the art to which this subject matter belongs.

Disclosed herein is a body cavity or wound drainage system having a reservoir into which body fluids can drain. The reservoir includes a convenient outlet assembly that can be opened or otherwise actuated to remove fluids or other contents from the reservoir such as when the reservoir is filled to capacity. The outlet assembly is configured such that the reservoir can be opened without cutting or otherwise damaging the reservoir. The material of the reservoir does not have to be cut, torn, bent or otherwise plastically deformed in order to open the reservoir.

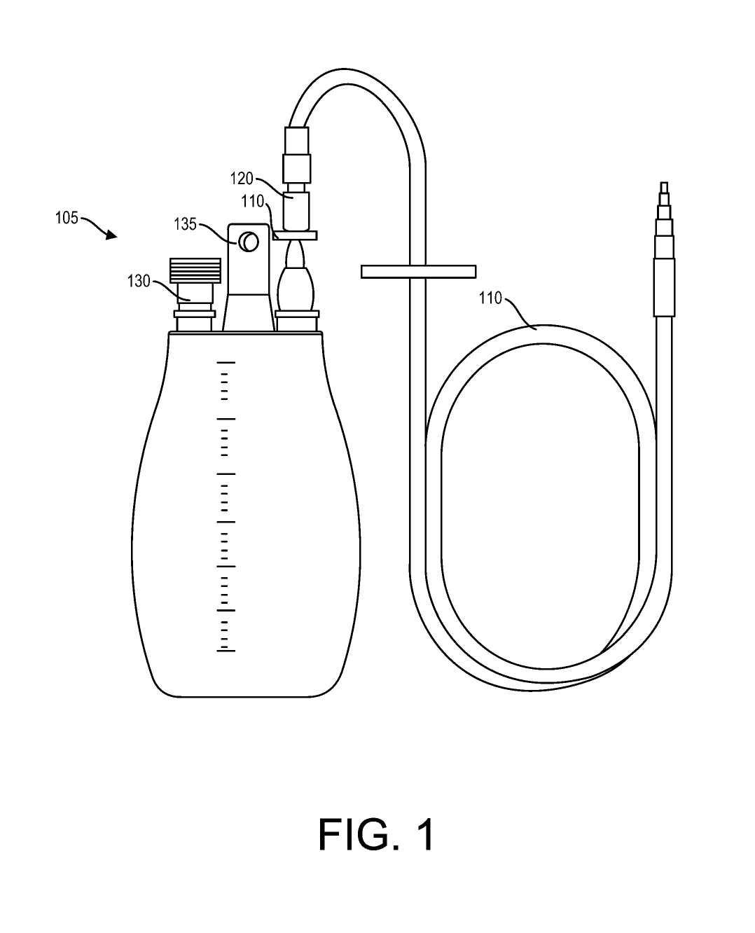

FIG. 1 shows a side view of an embodiment of an evacuatable or pre-evacuated reservoir, or container 105, which is part of a wound drainage system. The container is coupled to a drainage tube 110 that can be fluidly coupled to a body cavity for drainage of fluid of the body cavity into an internal cavity of the container 105 via the drainage tube 110. In this regard, the drainage tube 110 has an internal lumen that fluidly communicates with the internal cavity of the container 105 and with the body cavity. In an embodiment, the system is used pursuant to a Redon technique and the container is a Redon container. It should be appreciated that the system is not limited to use as part of a Redon technique and that it can be used for drainage of any of a variety of body fluid and body cavities.

With reference still to FIG. 1, the container 105 is formed of an outer body that defines and contains an internal cavity. The outer body can have any of a variety of shapes such as the bulbous shape shown in FIG. 1. The shape of the outer body can also be cylindrical as well as other shapes. In embodiment, the container 105 (such as the interior cavity) can be subjected, without problem, to a reduced pressure of up to or over 90,000 Pascals (90 kPa). The size of the container can vary. In example embodiments, the container is sized such that the interior cavity can contain up to 600 mL, 1,000 mL, or 1,500 mL of fluid. The outer body of the container can be made of various materials.

The container 105 may include one or more connection portions, such as a fluid interface 120 that removably or fixedly connects to the drainage tube 110 for evacuation of fluids into the container 105 via the drainage tube 110. The fluid interface 120 may include any type of connector for removably attaching to the drainage tube, such as a Luer lock for example. In an embodiment, the fluid interface 120 is made of a malleable material that can be clamped with a clamp device 125. The clamp device 125 occludes the fluid interface 120 when attached thereto to inhibit or completely prevent fluid flow through the fluid interface 120. In an embodiment, the fluid interface and/or the drainage tube include a check valve (such as a one-way valve) to prevent or inhibit backflow of fluid out of the container 105 via the fluid interface.

In addition, the container can include a pressure indicator 130 that provides an indication as to the fluid pressure state of the interior cavity. In an embodiment, the pressure indicator includes an expandable bellows or accordion-like structure that is initially in a default, contracted state when the interior of the container is under vacuum. The bellows structure is hollow. The hollow interior of the bellows communicates with the hollow interior of the container outer body. In an initial state, the container is under vacuum and the bellows is in the contracted state. When the container is opened and exposed to atmospheric pressure, such as via the fluid interface 120, the bellows expands in size to provide an indication that the vacuum has been released.

The container 105 can also have a suspension device 135 serves for suspending the container 105, for example, from an infusion stand or another mounting means for holding the container. The container 105 can also include markings, such as one or more graduated scales, on the outer body for providing an indication as to the fluid volume contained therein.

The container 105 must sometimes be emptied of fluid such as when the interior cavity is filled to capacity or for other reasons. In order to empty the container 105, a user must cut or otherwise destroy a portion of the container 105 or the drainage tube 110. For example, a user may cut the pressure indicator 130 at the bellows in order to form an opening into the interior cavity. It can be very cumbersome for a user to cut the container. There are now disclosed various embodiments of a biological fluid drainage container that includes outlet assemblies that can be easily actuated by a user to open the container and empty the container of its contents. As mentioned, a user does not have to cut, damage, tear, or otherwise deform the material of the container in order to open the container using the outlet assembly.

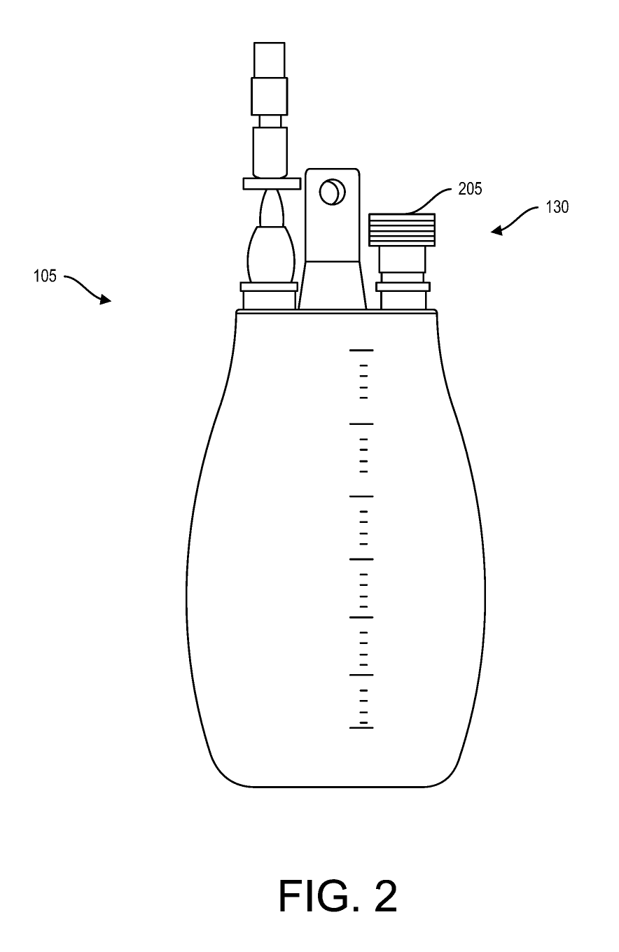

FIG. 2 shows a first embodiment of a container 105 that is similar or the same in size and shape as the previously described container 105 of FIG. 1. The container 105 of FIG. 2 includes a pressure indicator 130 in the form of a cylindrical body having an expandable bellows 205. The cylindrical body is hollow and positioned at the top of the container 105 although its position may vary. The hollow of the cylindrical body fluidly communicates with the interior cavity of the container 105. In a default state, the bellows 130 is in a contracted state such as when the container 105 is under vacuum. When the container 105 is placed in fluid communication with the body cavity via the drainage tube 110, the pressure inside the container increases thereby causing the bellows 130 to expand in shape (i.e., the expanded state) relative to the default state.

In this embodiment, the outlet assembly is formed of a threaded connection between the pressure indicator and the container body. That is, the pressure indicator 130 and its bellows 205 are removably attached to the container 105 via a threaded connection. The pressure indicator 130 may have a female, threaded feature or opening that receives a complementary male protrusion that can be threaded into the female feature. Or, the pressure indicator may have a threaded male feature that inserts into a complementary threaded, female feature of the container body. In this manner, the pressure indicator 130 can be screwed out of the body of the container 105 to form an opening between atmosphere and the interior cavity of the container 105. A user can then empty the contents of the container 105 through the opening that was formed by unscrewing the pressure indicator from the container body. It should be appreciated that other mechanical means aside from threads can be used to couple the pressure indicator to the container body. In addition, the pressure indicator 130 can be located at other locations on the container body aside from what is shown in FIG. 2.

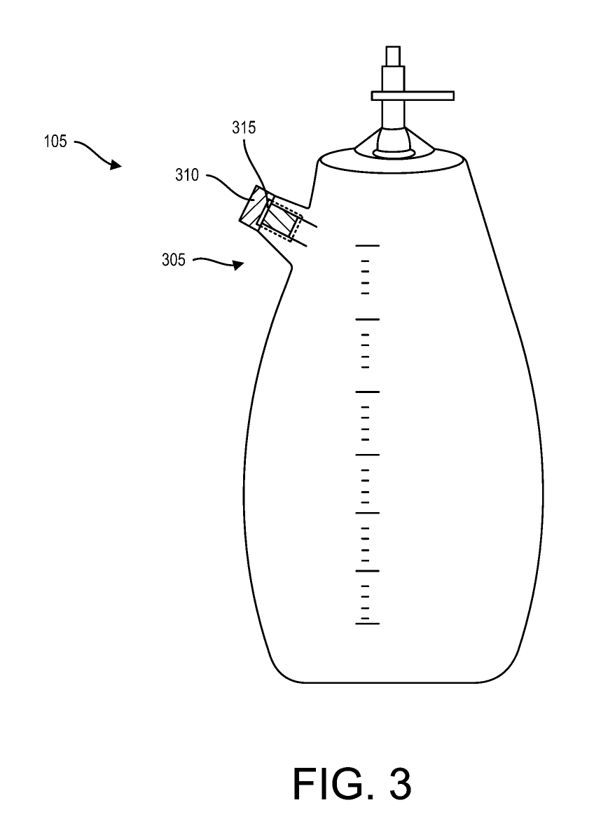

FIG. 3 shows another embodiment of a container 105. This embodiment has an outlet assembly formed of a spout 305 with an internal passageway that communicates with the interior of the container 105. The spout is located along an upper region of the container 105 although its position on the container can vary. A threaded plug 310 is removably positioned inside or on the spout 305 such that the plug blocks fluid flow through the spout when coupled thereto. A gasket 315 can be coupled to the spout such as around the plug 310 to provide a fluid seal between the plug and the internal passageway of the spout 305.

As mentioned, the plug 310 is threaded. The plug can be a male plug that inserts into the spout with complementary threads on the outer surface of the plug 310 and inner surface of the spout. Or the plug can be a female member that receives the outer surface of the spout 305 with complementary threads therebetween.

In use, a user can remove the plug 310 from the spout 305 by unthreading (such as by rotation) the plug 310 from the spout. Once the plug 310 is removed, an opening is formed between atmosphere and the interior of the container 105 via the internal passageway of the spout 305. This permits the use to empty the container and/or obtain a sample of the container contents via the opening.

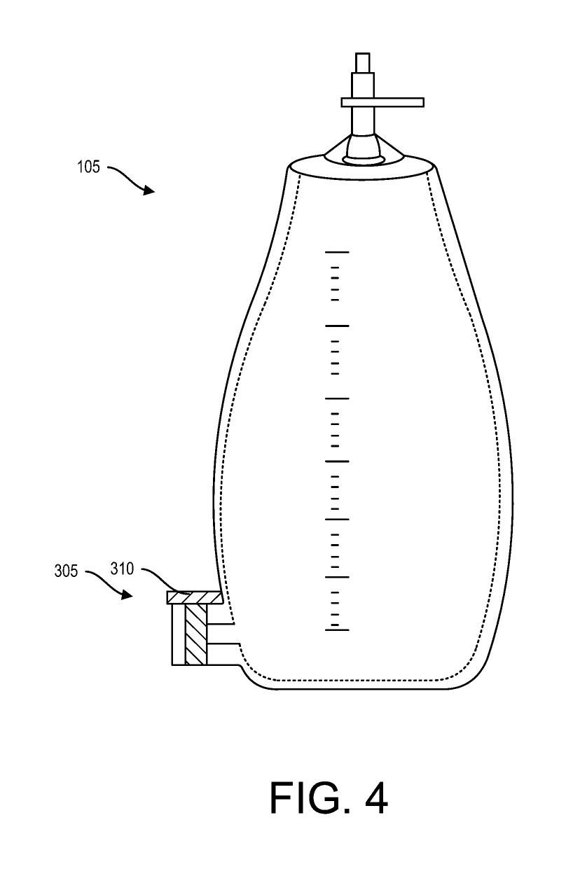

FIG. 4 shows another embodiment wherein the spout 305 is located at or near a bottom edge of the container 105. In the example embodiment, the spout 305 extends horizontally outward from the container body relative to the long, vertical axis of the container 105. Again, the spout has an internal passageway that communicates with the interior of the container 105. The plug 310 is coupled to the spout 305 such as in the previous embodiment. In this embodiment, the plug 310 is vertically oriented along a vertical axis and is rotated about the axis to unthread from the spout 305. Any of the embodiments of the plug 310 can be made of an expandable or pliable material.

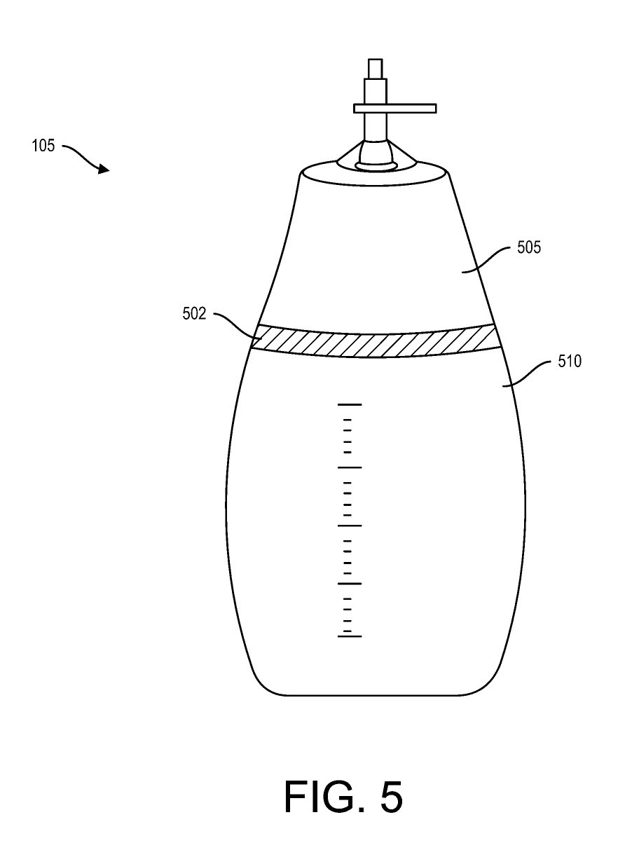

FIG. 5 shows another embodiment wherein the body of the container is made of two or more pieces that attach to one another along at least one seam 502 in a sealing relationship to collectively form a fluid tight, internal cavity. In this illustrated embodiment, the container 105 is formed of an upper portion 505 that forms an upper region of the container and a lower portion 510 that forms a lower portion of the container. The upper portion 505 removably attaches to the lower portion 510, such as in a male-female threaded relationship. In an embodiment, the lower portion 510 forms at least two third of the entire container. In a non-limiting example, the border or seam between the upper and lower portions is located at or above at least two third of the entire height of the container body. In an embodiment, at least one of the upper portion 505 and the lower portion 510 is a monolithic body.

In use, a user can remove the upper portion 505 from the lower portion such as by unthreading the upper portion from the lower portion. Once the upper portion is removed, the interior cavity is exposed for emptying and/or sampling of the contents.

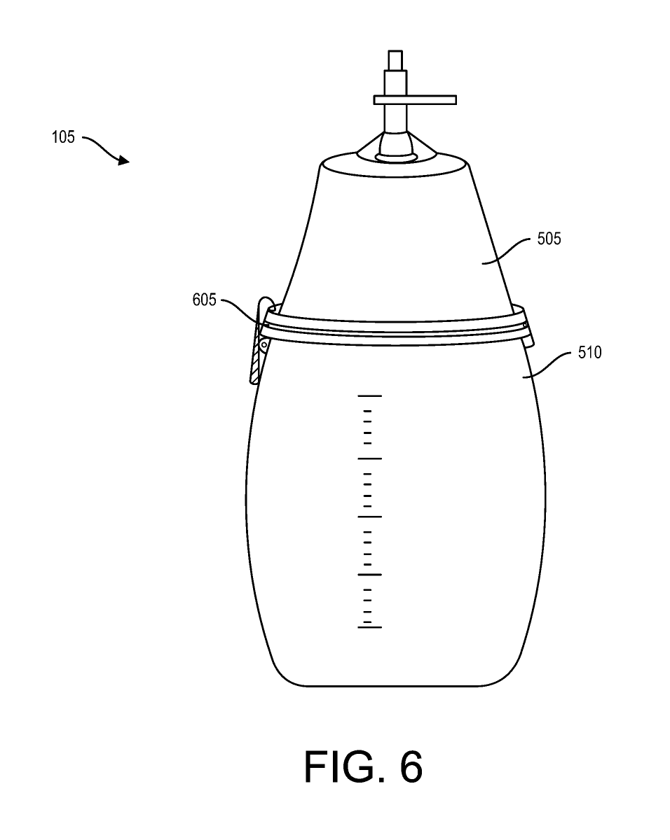

In another embodiment shown in FIG. 6, a locking clamp 605 is provided on the container 105. The locking clamp 605 is located at the seam between the upper portion 505 and lower portion 510 and it secures the upper portion 505 to the lower portion 510 in a fluid tight manner. The locking clamp 605 extends around the entire outer periphery of the container body at the seam. The locking clamp 605 can be any type clamp. In an example, the locking clamp 605 has a pivot joint that rotates a first portion of the locking clamp relative to a second portion of the locking clamp. The locking clamp 605 can be opened to release the upper portion 505 from the lower portion and expose the interior of the container 105 for emptying and/or sampling.

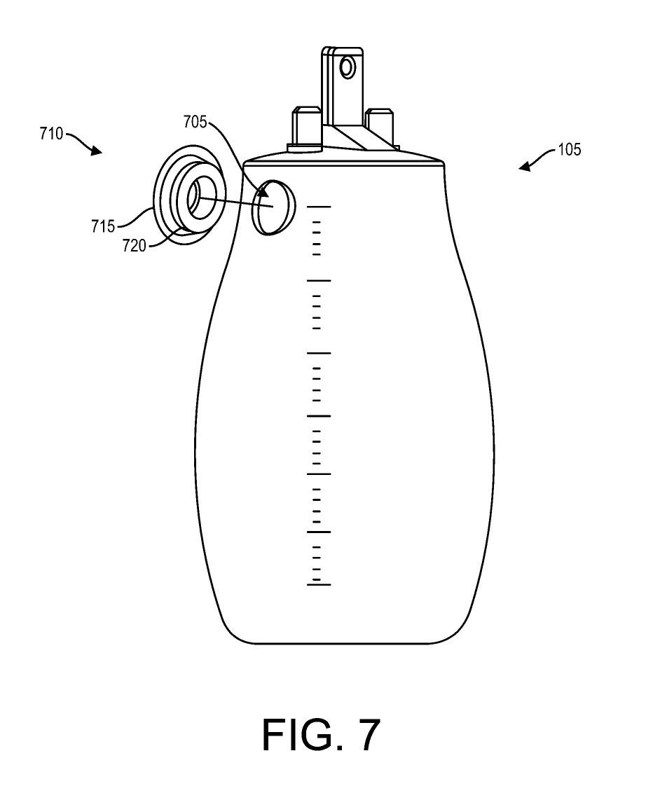

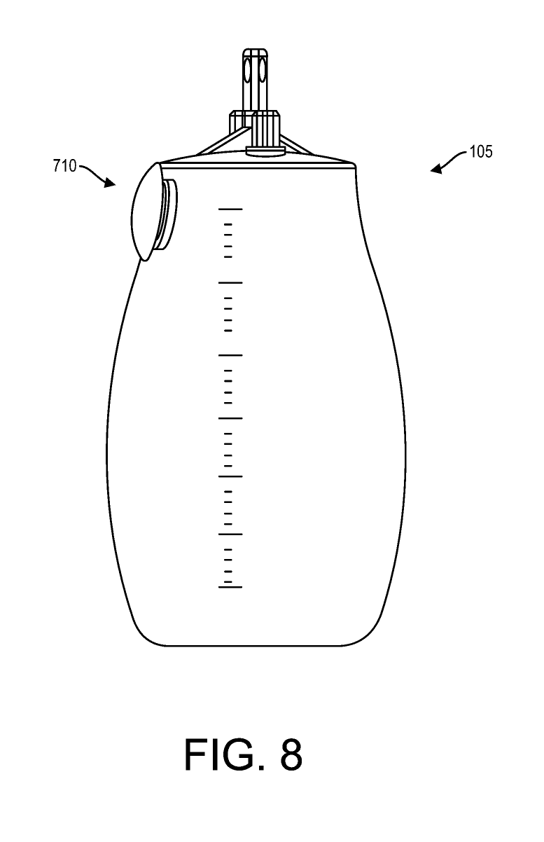

FIG. 7 shows yet another embodiment of a container 105. This embodiment includes an opening 705 that communicates with the interior of the container 105. A plug 710 is sized and shaped to fit at least partially inside the opening 705 in a manner that fluidly seals the opening 710. The plug 710 has a cap 715 that abuts the outer surface of the container 105 when the plug 710 is sealingly positioned in the opening 705. A male portion 720 of the plug 710 fits inside the opening 705 to seal the opening against passage of fluid therethrough. FIG. 8 shows the container with the plug 710 positioned inside the opening. The plug can be secured in the opening via a threaded connection between the plug and the opening. In another embodiment, the plug secures via a press fit between the plug and the opening.

In various implementations, description is made with reference to the figures. However, certain implementations may be practiced without one or more of these specific details, or in combination with other known methods and configurations. In the description, numerous specific details are set forth, such as specific configurations, dimensions, and processes, in order to provide a thorough understanding of the implementations. In other instances, well-known processes and manufacturing techniques have not been described in particular detail in order to not unnecessarily obscure the description. Reference throughout this specification to “one embodiment,” “an embodiment,” “one implementation, “an implementation,” or the like, means that a particular feature, structure, configuration, or characteristic described is included in at least one embodiment or implementation. Thus, the appearance of the phrase “one embodiment,” “an embodiment,” “one implementation, “an implementation,” or the like, in various places throughout this specification are not necessarily referring to the same embodiment or implementation. Furthermore, the particular features, structures, configurations, or characteristics may be combined in any suitable manner in one or more implementations.

The use of relative terms throughout the description may denote a relative position or direction. For example, “distal” may indicate a first direction away from a reference point. Similarly, “proximal” may indicate a location in a second direction opposite to the first direction. However, such terms are provided to establish relative frames of reference, and are not intended to limit the use or orientation of the systems to a specific configuration described in the various implementations.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

1. A pre-evacuated container for medical purposes, comprising:
an outer body that forms an internal cavity, wherein in a default state the internal cavity is at a vacuum pressure relative to atmosphere;
a fluid interface on the outer body that connects to a drainage tube such that fluids can be evacuated into the container from a body cavity via the drainage tube;
a pressure indicator on the outer body, the pressure indicator adapted to provide an indication as to a fluid pressure state of the internal cavity;
an outlet assembly on the outer body, wherein the outlet assembly can be actuated to form an opening between atmosphere and the internal cavity, wherein the outlet assembly can be actuated without damaging the outer body.

2. The container of claim 1, wherein the outlet assembly comprises a portion of the container that can be unthreaded from the outer body.

3. The container of claim 2, wherein the outlet assembly includes the pressure indicator.

4. The container of claim 1, wherein the pressure indicator is attached to the container body via a threaded connection and wherein the pressure indicator can be unthreaded and detached from the container body to form an opening between atmosphere and the internal cavity.

5. The container of claim 1, wherein the pressure indicator includes a bellows.

6. The container of claim 1, wherein the outlet assembly comprises a spout having an internal passageway that communicates with the internal cavity, and a plug that couples to the spout to block fluid flow through the internal cavity.

7. The container of claim 6, wherein the plug couples to the spout via a threaded connection.

8. The container of claim 6, wherein the spout is located at an upper region of the container body.

9. The container of claim 6, wherein the spout is located at a lower region of the container body.

10. The container of claim 1, wherein the outlet assembly comprises an upper portion of the container body and a lower portion of the container body, wherein the upper portion and lower portion are detachably connected to collectively form the internal cavity.

11. The container of claim 10, wherein a threaded interface connects the upper portion to the lower portion.

12. The container of claim 10, wherein a clamp removably secures the upper portion to the lower portion.

13. The container of claim 1, wherein the outlet assembly comprises an opening in the container body and a plug that removably couples to the opening to block fluid flow through the opening.

14. A pre-evacuated container for medical purposes, comprising:
an outer body that forms an internal cavity, wherein in a default state the internal cavity is at a vacuum pressure relative to atmosphere;
a fluid interface on the outer body that connects to a drainage tube such that fluids can be evacuated into the container from a body cavity via the drainage tube, wherein at least one of the fluid interface and the drainage tube includes a check valve that prevents back flow out of the internal cavity via the fluid interface;
a pressure indicator on the outer body, the pressure indicator adapted to provide an indication as to a fluid pressure state of the internal cavity;
an outlet assembly on the outer body, wherein the outlet assembly can be actuated to form an opening between atmosphere and the internal cavity, wherein the outlet assembly can be actuated without damaging the outer body.

15. The container of claim 14, wherein the outlet assembly comprises a portion of the container that can be unthreaded from the outer body.

16. The container of claim 15, wherein the outlet assembly includes the pressure indicator.

17. The container of claim 14, wherein the pressure indicator is attached to the container body via a threaded connection and wherein the pressure indicator can be unthreaded and detached from the container body to form an opening between atmosphere and the internal cavity.

18. The container of claim 14, wherein the pressure indicator includes a bellows.

19. The container of claim 14, wherein the outlet assembly comprises a spout having an internal passageway that communicates with the internal cavity, and a plug that couples to the spout to block fluid flow through the internal cavity.

20. The container of claim 14, wherein the outlet assembly comprises an upper portion of the container body and a lower portion of the container body, wherein the upper portion and lower portion are detachably connected to collectively form the internal cavity.
